(19) United States
(12) Patent Application Publication
(10) Pub. No.: US 2016/0331344 A1
(43) Pub. Date: Nov. 17, 2016

(54) ULTRASOUND PROBE COVER AND METHOD OF USE

(71) Applicant: Admir HADZIC, Weehawken, NJ (US)

(72) Inventor: Admir HADZIC, Weehawken, NJ (US)

(21) Appl. No.: 15/150,591

(22) Filed: May 10, 2016

Related U.S. Application Data
(60) Provisional application No. 62/162,374, filed on May 15, 2015.

(51) Int. Cl. A61B 8/00

(52) U.S. Cl. CPC A61B 8/4444; A61B 8/4281

(57) ABSTRACT

An ultrasound probe cover for use with an ultrasound probe comprises a sheath of material configured to substantially envelop the ultrasound probe and to transmit ultrasonic waves emitted by an insonating surface of the ultrasound probe. An insonating surface contacting region of the sheath comprises a contacting region inner surface and a contacting region outer surface. At least one barrier is removably attached to the contacting region inner surface, the contacting region outer surface, or both. Further, the at least one barrier comprises an indicia of a verification procedure to be performed prior to removal of the at least one barrier. A conducting medium may be disposed on the contacting region inner surface and/or the contacting region outer surface and covered by respective inner and/or outer barriers also disposed on the inner and/or outer surfaces.

### CROSS-REFERENCE TO RELATED APPLICATION

The instant application claims the benefit of Provisional U.S. Patent Application Ser. No. 62/162,374 entitled “ULTRASOUND TRANSDUCER COVER” and filed May 15, 2015, the teachings of which are incorporated herein by this reference.

### FIELD

The instant disclosure relates generally to ultrasound probes and, in particular, to an ultrasound probe cover and method for using such a cover.

### BACKGROUND

The practice of modern regional anesthesia (i.e., peripheral nerve blocks, spinal or regional anesthesia) is increasingly accomplished with aid of ultrasound imaging. As shown, for example, in FIG. 3, an ultrasound machine typically incorporates a probe or transducer 302 having a insonating surface 304 and a cable (not shown) connecting the probe to the ultrasound machine that, in turn, processes signals received from the probe 302 to generate and display the images essentially in real-time. Using high-frequency (ultrasonic), mechanical waves—typically in the 1-20 MHz range—emitted via the insonating surface 304 (which is placed in contact with the patient's skin), ultrasound imaging is able to construct images of internal structures of a patient based on reflections of the emitted ultrasonic waves.

Since ultrasound transducers are expensive, they are typically reusable. However, the practice of regional anesthesia or any other interventional ultrasound-guided procedure require sterile conditions because nerve blocks and/or interventional procedures typically involve introduction of a needle into the tissue. To accomplish sterile conditions, clinicians typically use disposable, sterile ultrasound probe covers to envelop the ultrasonic probe. Most available covers are made of a relatively thin, plastic- or rubber-like, transparent material that is capable of efficiently transmitting the ultrasonic sound waves with little attenuation or distortion. In addition to the sterility and infectious precautions, ultrasound probe covers also decrease the chance of blood cross-contamination between patients that might otherwise occur via blood-contaminated probes.

To facilitate obtaining optimal ultrasound images during ultrasound-guided nerve blocks (or other interventional procedures), clinicians often use a suitable conducting medium, such as ultrasound gel or adhesive, to eliminate occurrences of air between the insonating surface 304 of the ultrasound probe 302 and the sterile probe cover. An example of this is illustrated in FIGS. 1-4. In this example, an ultrasound probe cover 102 comprises a sheath of material 104 having an open end 106 and a closed end 108. As known in the art, all components of the ultrasound probe cover 102 are manufactured according to known procedures (e.g., Good Manufacturing Practice or GMP) to in order to ensure the proper configuration and sterility of the cover 102. The sheath 104 further comprises a insonating surface contacting region 110, which is generally configured to receive the insonating surface of the ultrasound probe. For example, the insonating surface contacting region 110 is preferably flat and without seams that might otherwise interfere with transmission of the ultrasonic waves. Furthermore, the insonating surface contacting region 110 comprises a contacting region outer surface 112, i.e., that surface coextensive with the insonating surface contacting region 110 on an outer side of the sheath 104, and a contacting region inner surface 114, i.e., that surface coextensive with the insonating surface contacting region 110 on an inner side of the sheath 104. As used herein, “inner” refers to those surfaces of the ultrasound probe cover that are contact with the ultrasound probe during use of the probe in a medical procedure, whereas “outer” refers to those surfaces of the ultrasound probe cover opposite the inner surfaces.

In the illustrated example, a barrier 118 is removably attached to the contacting region inner surface 114 and, in this case, an adhesive layer 116 is disposed between the barrier 118 and the contacting region inner surface 114. Techniques for providing such removable barriers, e.g., through use of suitable pressure-sensitive adhesives and releasable backing materials, are well known in the art. In use, as shown in FIG. 2, the ultrasound probe cover 102 is everted, typically around a sterilized hand 202 of a clinician that holds the ultrasound probe cover 102 in proximity to the closed end 108 and the insonating surface contacting region 110. In this everted state, the removable barrier 118 is exposed along with one or more tabs 204 that may be grasped to remove the barrier 118, thereby further exposing the adhesive layer 116. As shown in FIG. 3, with the barrier 118 removed and the adhesive layer 116 exposed, the insonating surface 304 of the probe 302 may be brought into contact with the adhesive layer such that adherence of the adhesive layer to the insonating surface results in substantially little or no air trapped between the insonating surface 304 and the everted sheath material 104′. Once the everted sheath 104′ and probe 302 are joined in this manner, the everted sheath 104′ is re-everted to return to its normal state such that that inner surfaces of the sheath 104 are in contact with the probe 302, as shown in FIG. 4. Though not illustrated in FIG. 4, the clinician or other team member will typically further secure the sheath 104 to the probe 302 and cable (not shown) by tightly bunching up the sheath and securing it in this position using a suitable rubber band, tape, etc. Additionally not shown in FIG. 4, clinicians will typically use an ultrasound gel disposed on the contacting region outer surface 112 (after the probe 302 has been enveloped by the ultrasound probe cover 104) before contacting the covered probe to the patient's skin. However, application of ultrasound gel to facilitate ultrasound imaging in a sterile manner usually requires an extra person to avoid contamination.

As further known in the art, during the performance of the peripheral nerve blocks or any other invasive interventional procedure, there is a constant danger that the procedure may be mistakenly performed on the wrong side of the patient's body, for instance, on the wrong extremity. To minimize this possibility, however, most hospitals and medical practices now mandate a verification or “time out” procedure, including documentation by the medical team of the proper extremity or surgical site, procedure, laterality and patient identification. However even with these mandatory provisions, procedures on the wrong side of the body or wrong extremity continue to occur, thereby placing tens of thousands of patients at risk from associated morbidity and mortality, at a total estimated cost in the tens of billions of dollars each year.

Recent data suggest that wrong-site nerve blocks are actually more common that wrong-site surgeries. In addition to potentially increasing the length and cost of hospital stay, wrong-site nerve block procedures unnecessarily expose patients to various block-related complications, which can have serious consequences. The most common reason for continued wrong-site surgeries and nerve block procedures, despite the mandatory verification procedures, is the fact that care teams still often rely on memory to implement the verification or time out procedure before performing the actual surgical procedure.

Thus, it would be advantageous to provide ultrasound probe covers and procedures for use thereof that overcome the above-noted deficiencies of prior art techniques.

### SUMMARY

The instant disclosure describes an ultrasound probe cover for use with an ultrasonic probe. In an embodiment, an ultrasound probe cover comprises a sheath of material that is configured to substantially envelop the ultrasound probe and to transmit ultrasonic waves emitted by a insonating surface of the ultrasound probe. The sheath has an open end, a closed end and a insonating surface contacting region. The insonating surface contacting region, in turn, comprises a contacting region inner surface and a contacting region outer surface. In an embodiment, the insonating surface contacting region is proximate the closed end of the sheath. At least one barrier is removably attached to the contacting region inner surface, the contacting region outer surface, or both. Further, the at least one barrier comprises an indicia of a verification procedure to be performed prior to removal of the at least one barrier.

In an embodiment, a conducting medium is disposed on the contacting region inner surface. The conducting medium may comprise an ultrasonic gel or adhesive. In this embodiment, the at least one barrier may comprise an inner barrier disposed on the contacting region inner surface such that the conducting medium is disposed between the contacting region inner surface and the inner barrier.

In another embodiment, a conducting medium is disposed on the contacting region outer surface, which conducting medium may comprise an ultrasonic gel. In this embodiment, the at least one barrier may comprise an outer barrier disposed on the contacting region outer surface such that the conducting medium is disposed between the contacting region outer surface and the outer barrier.

Related methods are also disclosed herein.

### BRIEF DESCRIPTION OF THE DRAWINGS

The features described in this disclosure are set forth with particularity in the appended claims. These features and attendant advantages will become apparent from consideration of the following detailed description, taken in conjunction with the accompanying drawings. One or more embodiments are now described, by way of example only, with reference to the accompanying drawings wherein like reference numerals represent like elements and in which:

FIGS. 1-4 are schematic illustrations of an ultrasound probe cover, shown in partial cross-section, for use in conjunction with an ultrasound probe in accordance with prior art techniques and further illustrating use of the surgical drape;

FIGS. 5 and 6 are illustrations of an everted ultrasound probe cover in accordance with the instant disclosure, particularly illustrating indicia of a verification procedure included on at least one barrier and further illustrating use of such ultrasound probe cover;

FIGS. 7-9 are schematic illustrations of other embodiments of everted ultrasound probe covers, shown in partial cross-section, in accordance with the instant disclosure; and

FIG. 10 illustrates a flow chart depicting a method of using an ultrasound probe cover in accordance with the instant disclosure.

### DETAILED DESCRIPTION OF THE PRESENT EMBODIMENTS

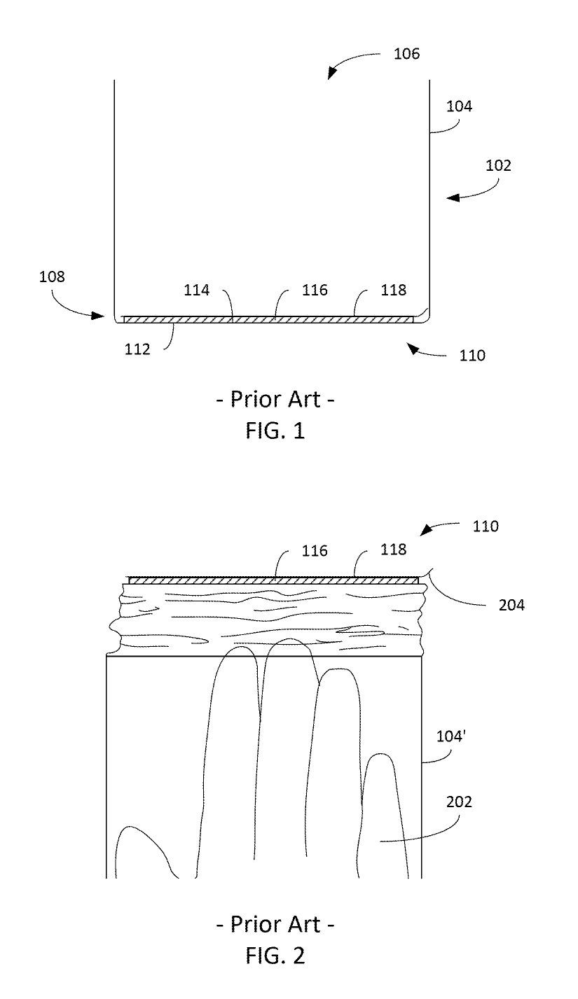

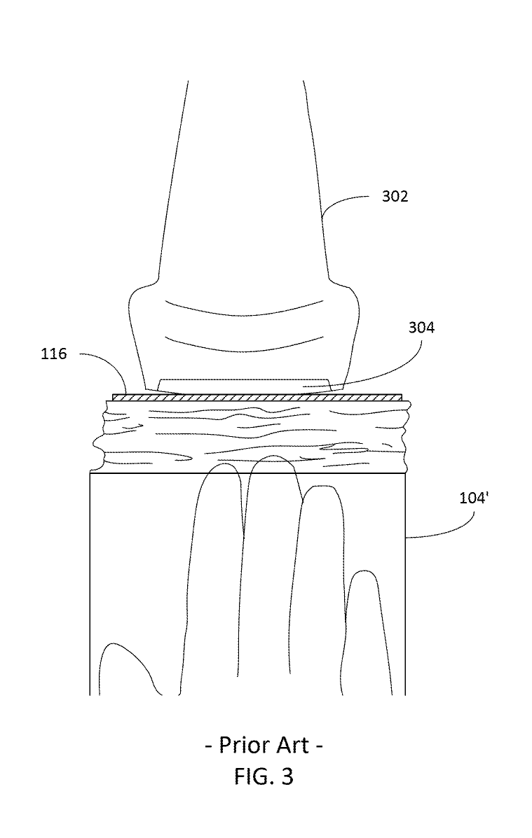

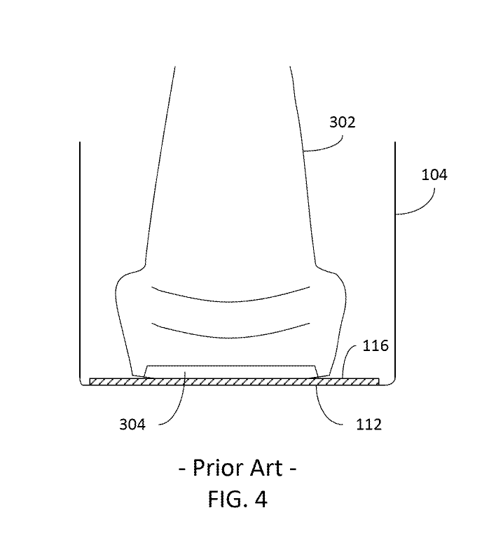

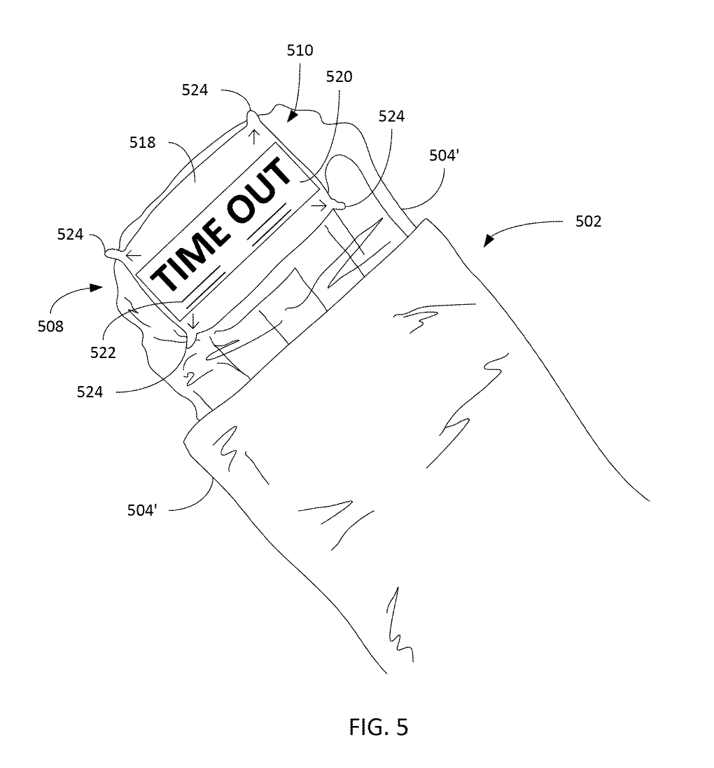

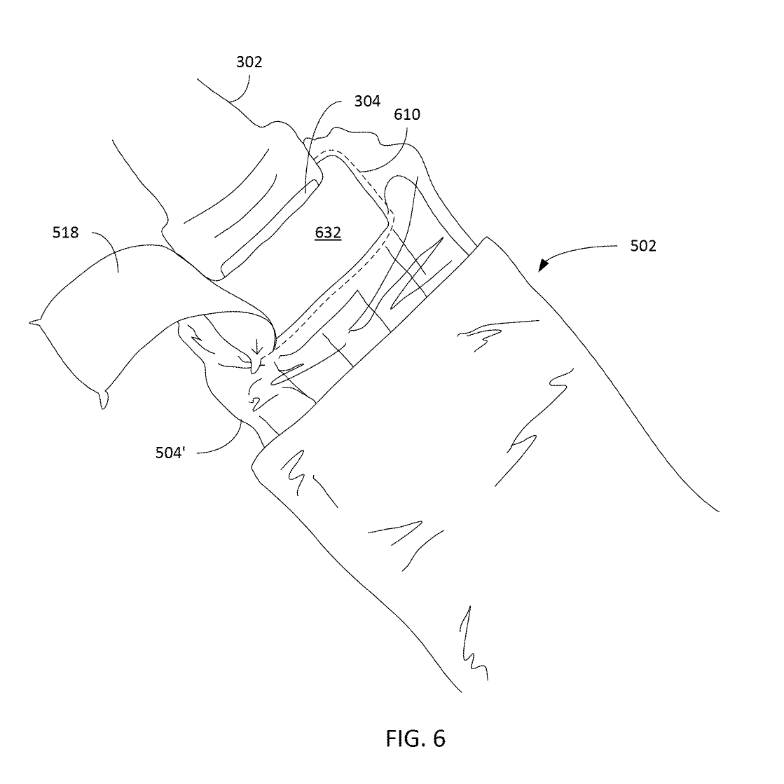

Referring now to FIGS. 5 and 6, a ultrasound probe cover 502 in accordance with an embodiment of the instant disclosure is illustrated. In particular, the ultrasound probe cover 502 is illustrated in an everted state, similar to FIGS. 2 and 3, described above, in which a clinician holds the everted sheath 504 proximate the closed end 508 and, more particularly, the insonating surface contacting region 510. Similar to the cover 102 illustrated in FIGS. 1-4, the ultrasound probe cover 502 of FIGS. 5 and 6 includes an inner barrier 518 removably attached to an inner surface 610 of the insonating surface contacting region. In the illustrated example, the inner barrier 518 comprise several tabs 524 that may be grasped to facilitate removal of the inner barrier 510.

As shown in FIG. 5, the inner barrier 518, which may be manufactured from a suitable paper or plastic materials or lamination of such suitable materials, further comprises indicia 520 of a verification procedure and, optionally, laterality (left or right) of the procedures, as described above, to be performed prior to removal of the inner barrier 518. In the illustrated example, the indicia 520 of the verification procedure comprises the words “TIME OUT” in a large, easily-viewable font. Those having skill in the art will appreciate that other features of the inner barrier 518 may be used as the indicia 520 including, for example, the color of the font or background thereof, the degree of contrast between the font and its background, etc. Further still, different text and/or an image may be used as the indicia 520, or the presence of another material or substance that would otherwise prevent performance of ultrasound imaging and therefore require prior removal. As further shown, the inner barrier may further comprise lines or ruling 522 that may be used to receive an indication that performance of the verification procedure has been confirmed, such as one or more signatures of medical team members, a time at which the verification procedure was performed, information identifying the patient, surgical site, extremity and/or laterality, etc. Further still, the lines or ruling 522 may be used for technical information concerning the procedure performed, e.g., the stimulating current employed, type of needle employed, etc.

In order to perform the surgical procedure, the inner barrier 518 must first be removed as illustrated in FIG. 6. As shown in FIG. 6, the inner barrier 518 may be peeled back to expose a conducting medium 632, for example, in this case, a layer of suitable adhesive disposed on the inner surface of the contacting region 610. Once the conducting medium 632 is thus exposed, the insonating surface 304 of the ultrasound probe 302 may be brought into contact with the conducting medium 632. Thereafter, the everted sheath 504′ may be re-everted to envelop the probe 302, thereby maintaining sterile conditions. When the inner barrier 518 is completely removed, it may be placed in a patient's medical records as evidence that the verification procedure was performed prior to use of the ultrasound probe 302.

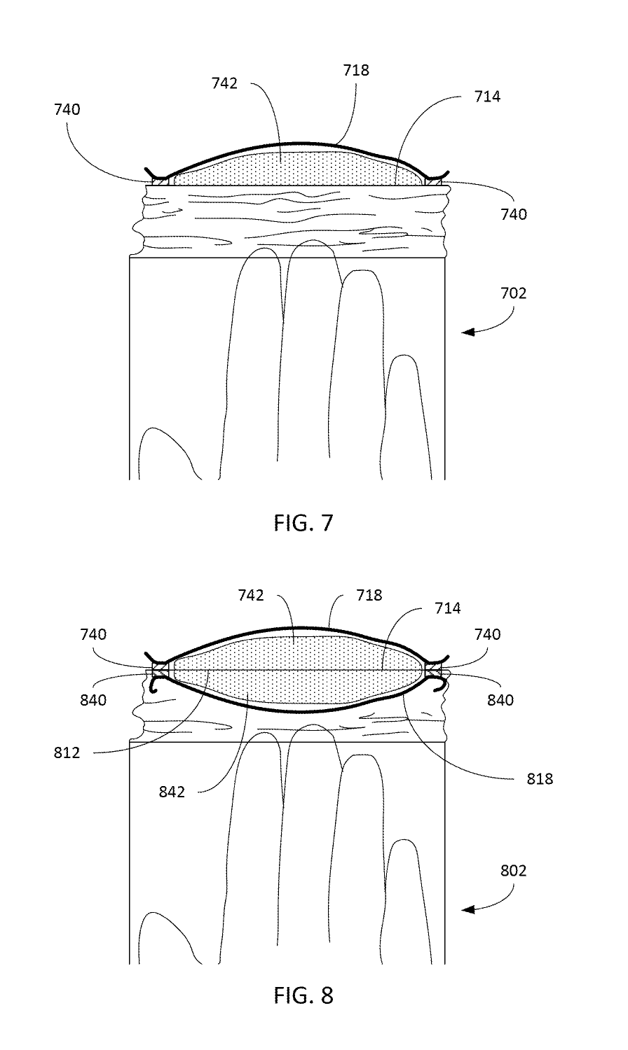

Referring to FIG. 7, an embodiment of a ultrasound probe cover 702 substantially similar to the embodiment of FIG. 6 is further illustrated. In particular, the conducting medium 742 illustrated in FIG. 7, instead of an adhesive, comprises ultrasound gel disposed between the contacting region inner surface 714 and the inner barrier 718 (also comprising the indicia 520 of the verification procedure; not shown). Those having skill in the art will appreciate that any of a number of suitable ultrasonic gels or other ultrasound-coupling medium (e.g., water, oil, etc.) may be used for this purpose. In this embodiment, the inner barrier 718 is attached to the contacting region inner surface 714 by an annular ring of adhesive 740, thereby forming a pocket or pouch having the ultrasound gel 742 disposed therein. The adhesive 740 may comprise a suitable pressure-sensitive adhesive having sufficient strength of adhesion to retain the ultrasound gel 742 within the pocket, while still permitting relatively easy removal of the inner barrier 718. Once the inner barrier is removed, the ultrasound gel 742 is exposed, thereby permitting the insonating surface 304 of an ultrasound probe 302 (not shown) to be inserted into the gel 742. An advantage of this embodiment is that it facilities one-person deployment of the ultrasound probe cover 702, i.e., it does not require an assistant to dispose the ultrasound gel on the drape 702 in order to maintain sterility. Additionally, other techniques for forming such a pocket in which removal of the barrier 718 results in exposure of the ultrasound gel may be equally employed, e.g., ultrasonic welding of the barrier to the sheath, etc.

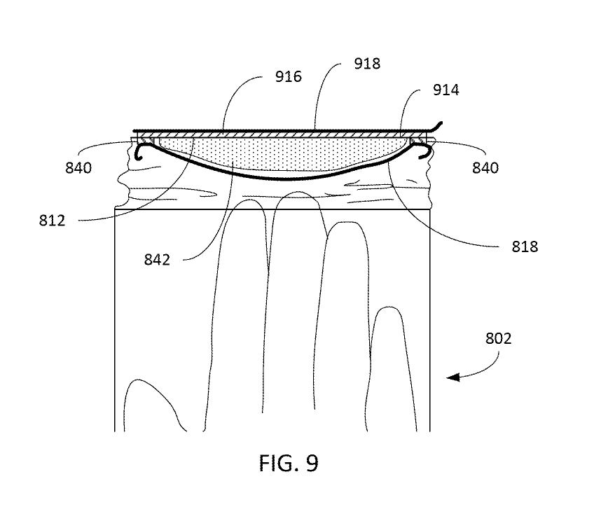

FIGS. 8 and 9 illustrate embodiments in which ultrasound probe covers 802, 902 are provided with an outer barrier 818 removably attached to a contacting region outer surface 812 with a conducting medium 842 disposed therebetween. In these embodiments, the conducting medium 842 comprises ultrasound gel 842. Similar to the embodiment of FIG. 7, the outer barrier 818 shown in FIGS. 8 and 9 is attached to the contacting region outer surface 812 by an annular ring of adhesive 840, as described above, thereby forming a pocket or pouch having the ultrasound gel 842 disposed therein. Once again, the outer barrier 818 shown in FIGS. 8 and 9 includes the indicia 520 of the verification procedure (not shown).

In the embodiment of FIG. 8, the outer barrier 818 and ultrasound gel 842 disposed on the contacting region outer surface 812 is combined with the inner barrier 718 and ultrasound gel 742 disposed on the contacting region inner surface 714. Because both the inner barrier 718 and the outer barrier 818 comprise the indicia 520 of the verification procedure, which barriers must be removed prior to using the ultrasound probe (not shown), the resulting system provides redundant reminders to perform the verification procedure thereby enhancing the likelihood that the verification procedure will be properly performed. Additionally, disposition of the ultrasound gel 742, 842 on both the contacting region inner surface 714 and contacting region outer surface 812 beneath the respective barriers 718, 818 eliminates the need to separately dispense a conducting medium on the sheath, thereby facilitating use of the cover 802.

In the embodiment of FIG. 9, the outer barrier 818 and ultrasound gel 842 disposed on the contacting region outer surface 812 is combined with an inner barrier 918 and an adhesive layer 916 (equivalent to the inner barrier 518 and adhesive layer 632 illustrated in FIGS. 5 and 6, described above) disposed on the contacting region inner surface 914. Once again, because both the inner barrier 918 and the outer barrier 818 comprise the indicia 520 of the verification procedure, redundant reminders enhancing the likelihood that the verification procedure will be properly performed. According to this embodiment, the benefits of having conducting media disposed on both the contacting region inner layer 914 and the contacting region outer layer 812 are retained, but in this instance, the ultrasound gel disposed on the contacting region inner layer 914 is replaced by the adhesive layer 916, thereby accommodating preferences of clinicians that may prefer this approach.

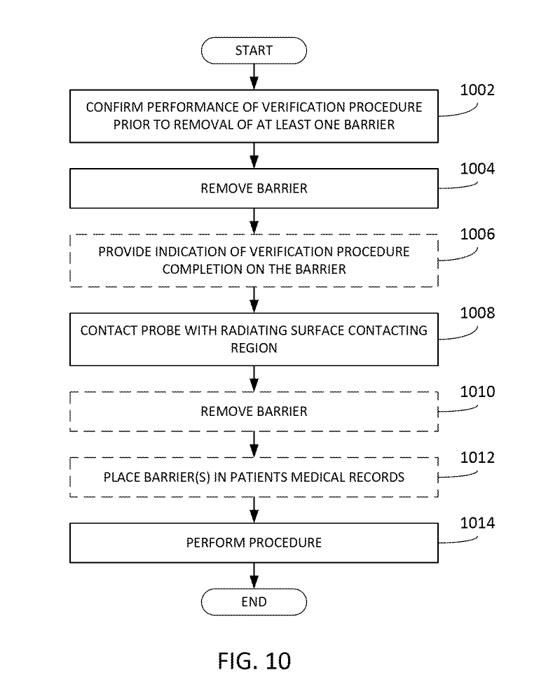

Referring now to FIG. 10, a method for using ultrasound probe covers as described herein is further illustrated. Thus, at block 1002, a clinician or medical team member first confirms performance of the verification procedure prior to removal of the at least one barrier 518, 718, 818, 918. Once the confirmation of verification procedure compliance has been completed, one or more of the barriers may be removed at block 1004. For example, an inner barrier 518, 718, 918 may be removed. Additionally, at block 1006, an indication of completion of the verification procedure may be optionally provided on the barrier as in the case, for example, where a clinician or medical team member employs the lines or rulings 522 to write down such information on the barrier.

Regardless, at block 1008, the ultrasonic probe is contacted with the insonating surface contacting region of the ultrasound probe cover that, as described above, may comprise a conducting medium disposed on a surface of the insonating surface contacting region and beneath the previously-removed barrier. If an additional barrier is present, as in the case of an outer barrier 818, such additional barrier may be removed at block 1010. Once again, any desired information concerning the completion of the verification procedure may be included on the additional barrier. Regardless of the number of barriers employed, the removed barriers may be optionally placed in the patient's medical records at block 1012, thereby serving as documentation of the completion of the verification procedure. Thereafter, having removed all barriers from the surgical drape, the desired surgical procedure incorporating the ultrasound probe cover may be performed at block 1014.

As described above, various embodiments of ultrasound probe covers have been described that facilitate their use with ultrasound probes and better ensure adherence to pre-surgery verification procedures. This is achieved through provision of indicia of the verification procedure on barrier disposed on the surgical drape, which barriers prevent use of the drape with an ultrasound probe prior to removal of the barriers. Furthermore, conducting media may be disposed between one or more barriers and a surface of a contacting region of the drape to which the barriers are attached. For at least these reasons, the above-described techniques represent an advancement over prior art teachings.

While particular preferred embodiments have been shown and described, those skilled in the art will appreciate that changes and modifications may be made without departing from the instant teachings. It is therefore contemplated that any and all modifications, variations or equivalents of the above-described teachings fall within the scope of the basic underlying principles disclosed above and claimed herein.

What is claimed is:

1. An ultrasound probe cover for use with an ultrasound probe comprising a insonating surface, the ultrasound probe cover comprising:
a sheath of material configured to substantially envelop the ultrasound probe and to transmit ultrasonic waves emitted by the insonating surface, the sheath having an open end and a closed end and an insonating surface contacting region, the insonating surface contacting region comprising a contacting region inner surface and a contacting region outer surface; and
at least one barrier removably attached to the contacting region inner surface or the contacting region outer surface or both, the at least one barrier comprising indicia of a verification procedure to be performed prior to removal of the at least one barrier.

2. The ultrasound probe cover of claim 1, further comprising:
a conducting medium disposed on the contacting region inner surface.

3. The ultrasound probe cover of claim 2, wherein the conducting medium comprises either an adhesive or an ultrasonic gel.

4. The ultrasound probe cover of claim 2, wherein the at least one barrier comprises an inner barrier disposed on the contacting region inner surface, and wherein the conducting medium is disposed between the contacting region inner surface and the inner barrier.

5. The ultrasound probe cover of claim 1, further comprising:
a conducting medium disposed on the contacting region outer surface.

6. The ultrasound probe cover of claim 5, wherein the conducting medium comprises an ultrasonic gel.

7. The ultrasound probe cover of claim 5, wherein the at least one barrier comprises an outer barrier disposed on the contacting region outer surface, and wherein the conducting medium is disposed between the contacting region outer surface and the outer barrier.

8. The ultrasound probe cover of claim 1, wherein the insonating surface contacting region is proximate the closed end.

9. A method for using an ultrasound probe cover in conjunction with an ultrasonic probe comprising an insonating surface, the method comprising:
contacting the ultrasonic probe with an insonating surface contacting region of the surgical drape, the ultrasound probe cover comprising a sheath of material configured to substantially envelop the ultrasound probe and to transmit ultrasonic waves emitted by the insonating surface, the sheath having an open end and a closed end and the insonating surface contacting region, the insonating surface contacting region comprising a contacting region inner surface and a contacting region outer surface, the ultrasound probe cover further comprising at least one barrier removably attached to the contacting region inner surface or the contacting region outer surface or both, the at least one barrier comprising indicia of a verification procedure to be performed prior to removal of the at least one barrier;
confirming performance of the verification procedure prior to removal of the at least one barrier; and
removing the at least one barrier prior to performing a surgical procedure using the ultrasonic probe.

10. The method of claim 9, wherein confirming performance of the verification procedure includes confirming at least one of a correct surgical site, a correct surgical procedure, a correct patient, and a correct laterality of the patient.

11. The method of claim 9, further comprising:
providing an indication on the barrier that the verification procedure has been confirmed.

12. The method of claim 9, further comprising:
after removing the at least one barrier, placing the barrier in a patient's medical records.
